(19) United States
(12) Patent Application Publication
(10) Pub. No.: US 2019/0112383 A1
(43) Pub. Date: Apr. 18, 2019

(54) TREATMENT OF B-CELL LYMPHOMA

(71) Applicant: BAYER PHARMA AKTIENGESELLSCHAFT, Berlin (DE)

(72) Inventors: Werner Krause, Berlin (DE); Joachim Kalmus, Berlin (DE); Jens Kuhlmann, Berlin (DE)

(21) Appl. No.: 15/978,460

(22) Filed: May 14, 2018

Related U.S. Application Data
(63) Continuation of application No. 11/176,671, filed on Jul. 8, 2005, now abandoned.
(60) Provisional application No. 60/586,414, filed on Jul. 9, 2004.

(51) Int. Cl. C07K 16/28; A61K 45/06; A61K 31/7072; A61K 51/10; A61K 31/704; A61K 41/00; A61K 39/395

(52) U.S. Cl. CPC C07K 16/2887; A61K 45/06; A61K 31/7072; A61K 2039/505; A61K 31/704; A61K 41/00; A61K 39/395; A61K 51/1027

(57) ABSTRACT

A method of treating B-cell lymphoma comprises administering to a patient a chemotherapeutic regimen, followed by treatment with a radiolabeled anti-CD20 antibody, wherein at the time of said treatment with said radiolabeled antibody said patient is not refractory to said chemotherapeutic regimen and has not relapsed.

This application claims the benefit of the filing date of U.S. Provisional Application Ser. No. 60/586,414 filed Jul. 9, 2004, which is incorporated by reference herein.

### FIELD OF THE INVENTION

The present invention relates to the treatment of patients newly diagnosed with B-cell lymphoma and not previously treated or previously treated and responding to chemotherapy with or without adding anti-CD20 antibody. The invention involves adding to chemotherapy (with or without anti-CD20 antibody) a radiolabeled anti-CD20 antibody.

### BACKGROUND OF THE INVENTION

Non-Hodgkin's lymphomas (NHLs) are a heterogeneous group of lymphoproliferative malignancies with differing patterns of behavior and responses to treatment.[NCI website; N Engl J Med 328 (14): 1023-30, 1993].

Like Hodgkin's lymphoma, NHL usually originates in lymphoid tissues and can spread to other organs. However, NHL is much less predictable than Hodgkin's lymphoma and has a far greater predilection to disseminate to extranodal sites. The prognosis depends on the histologic type, stage, and treatment.

The NHLs can be divided into 2 prognostic groups: the indolent lymphomas and the aggressive lymphomas. Indolent NHL types have a relatively good prognosis, with median survival as long as 10 years, but they usually are not curable in advanced clinical stages. Early-stage (I and II) indolent NHL can be effectively treated with radiation therapy alone. Most of the indolent types are nodular (or follicular) in morphology. The aggressive type of NHL has a shorter natural history, but a significant number of these patients can be cured with intensive combination chemotherapy regimens. In general, with modern treatment of patients with NHL, overall survival at 5 years is approximately 50% to 60%. Thirty percent to 60% of patients with aggressive NHL can be cured. The vast majority of relapses occur in the first 2 years after therapy. The risk of late relapse is higher in patients with a divergent histology of both indolent and aggressive disease.[Blood 79 (4): 1024-8, 1992].

While indolent NHL is responsive to radiation therapy and chemotherapy, a continuous rate of relapse is usually seen in advanced stages. However, patients can often be retreated with considerable success as long as the disease histology remains low grade. Patients who present with or convert to aggressive forms of NHL may have sustained complete remissions with combination chemotherapy regimens or aggressive consolidation with marrow or stem cell support.[J Clin Oncol 15 (4): 1587-94, 1997; J Clin Oncol 13 (7): 1726-33, 1995].

Radiation techniques differ somewhat from those used in the treatment of Hodgkin's lymphoma. The dose of radiation therapy usually varies from 2,500 cGy to 5,000 cGy and is dependent on factors that include the histologic type of lymphoma, the patient's stage and overall condition, the goal of treatment (curative or palliative), the proximity of sensitive surrounding organs, and whether the patient is being treated with radiation therapy alone or in combination with chemotherapy. Given the patterns of disease presentations and relapse, treatment may need to include unusual sites such as Waldeyer's ring, epitrochlear, or mesenteric nodes. However, the associated morbidity of the treatment must be considered carefully. The majority of patients who receive radiation are usually treated on only 1 side of the diaphragm. Localized presentations of extranodal NHL may be treated with involved-field techniques with significant (>50%) success.

In asymptomatic patients with indolent forms of advanced NHL, treatment may be deferred until the patient becomes symptomatic as the disease progresses. When treatment is deferred, the clinical course of patients with indolent NHL varies; frequent and careful observation is required so that effective treatment can be initiated when the clinical course of the disease accelerates. Some patients have a prolonged indolent course, but others have disease that rapidly evolves into more aggressive types of NHL that require immediate treatment.

Aggressive lymphomas are increasingly seen in HIV-positive patients; treatment of these patients requires special consideration.

Indolent (follicular) lymphoma comprises 20% of all non-Hodgkin's lymphomas and up to 70% of the indolent lymphomas reported in American and European clinical trials.[J Clin Oncol 16 (8): 2780-95, 1998; Blood 89 (11): 3909-18, 1997; Am J Surg Pathol 21(1): 114-121, 1997]. Most patients with follicular lymphoma are over age 50 and present with widespread disease at diagnosis. Nodal involvement is most common, often accompanied by splenic and bone marrow disease. Rearrangement of the bcl-2 gene is present in over 90% of patients with follicular lymphoma; overexpression of the bcl-2 protein is associated with the inability to eradicate the lymphoma by inhibiting apoptosis.[Blood 93 (9): 3081-7, 1999].

Despite the advanced stage, the median survival ranges from 8 to 12 years, leading to the designation of being “indolent.”[Oncology 54 (6): 441-58, 1997; Brit. J Clin Oncol 21 (1): 5-15, 2003]. However, the vast majority of patients with advanced-stage follicular lymphoma are not cured with current therapeutic options. The rate of relapse is fairly consistent over time, even in patients who have achieved complete responses to treatment. Watchful waiting, deferring treatment until the patient becomes symptomatic, is an option for patients of advanced stage [Lancet 362 (9383): 516-22, 2003].

Follicular small cleaved cell lymphoma and follicular mixed small cleaved and large cell lymphoma do not have reproducibly different disease-free or overall survivals.[R.E.A.L. to W.H.O. and beyond. Cancer: Principles and Practice of Oncology Updates 13(3): 1-14, 1999]. Therapeutic options include watchful waiting, purine nucleoside analogs, oral alkylating agents, combination chemotherapy, interferon, and monoclonal antibodies [Semin Oncol 26 (5 Suppl 14): 2-11, 1999]. Radiolabeled monoclonal antibodies, vaccines, and autologous or allogeneic bone marrow or peripheral stem cell transplantation are under clinical evaluation.[Semin Oncol 26 (5 Suppl 14): 2-11, 1999].

Aggressive (diffuse large B-cell) lymphoma is the most common of the non-Hodgkin's lymphomas, comprising 30% of newly-diagnosed cases [J Clin Oncol 16 (8): 2780-95, 1998]. Most patients present with rapidly enlarging masses, often with symptoms both locally and systemically (designated B symptoms with fever, recurrent night sweats, or weight loss). The vast majority of patients with localized disease are curable with combined modality therapy.[N Engl J Med 339 (1): 21-6, 1998]. For patients with advanced-stage disease, 40% of presenting patients are cured with doxorubicin-based combination chemotherapy [N Engl J Med 328 (14): 1002-6, 1993].

Treatment of non-Hodgkin's lymphoma (NHL) depends on the histologic type and stage. Many of the improvements in survival have been made using clinical trials (experimental therapy) that have attempted to improve on the best available accepted therapy (conventional or standard therapy).

Even though standard treatment in patients with lymphomas can cure a significant fraction, numerous clinical trials that explore improvements in treatment are in progress. If possible, patients should be included in these studies. Standardized guidelines for response assessment have been suggested for use in clinical trials.[J Clin Oncol 17 (4): 1244, 1999].

Late effects of treatment of NHL have been observed. Pelvic irradiation and large cumulative doses of cyclophosphamide have been associated with a high risk of permanent sterility [Clin Oncol 11 (2): 239-47, 1993]. For up to 2 decades after diagnosis, patients are at significantly elevated risk of second primary cancers, especially lung, brain, kidney, and bladder cancers and melanoma, Hodgkin's lymphoma, and acute nonlymphocytic leukemia [J Natl Cancer Inst 85 (23): 1932-7, 1993]. Left ventricular dysfunction was a significant late effect identified in 8 of 57 long-term survivors of high-grade NHL who received more than 200 milligrams per meter squared of doxorubicin [J Clin Oncol 16 (6): 2070-9, 1998]. Myelodysplastic syndrome and acute myelogenous leukemia are late complications of myeloablative therapy with autologous bone marrow or peripheral blood stem cell support, as well as conventional chemotherapy-containing alkylating agents [J Clin Oncol 12 (12): 2527-34, 1994; J Clin Oncol 21 (5): 897-906, 2003; Blood 103 (4): 1222-8, 2004]; most of these patients show clonal hematopoiesis even before the transplantation, suggesting that the hematologic injury usually occurs during induction or reinduction chemotherapy [J Clin Oncol 21 (5): 897-906, 2003; Blood 91 (12): 4496-503, 1998; J Clin Oncol 19 (9): 2472-81, 2001]. Successful pregnancies with children born free of congenital abnormalities have been reported in young women after autologous bone marrow transplantation [Leuk Lymphoma 28 (1-2): 127-32, 1997].

Although localized presentations are uncommon in non-Hodgkin's lymphoma (NHL), the goal of treatment should be cure in those who are shown to have truly localized disease after undergoing appropriate staging procedures. Long-term disease control within radiation fields can be achieved in a significant number of patients with indolent stage I or stage II NHL by using doses of radiation that usually range from 2500 to 4000 cGy to involved sites or to extended fields which cover adjacent nodal sites [J Clin Oncol 21 (13): 2474-80, 2003]. The value of adjuvant chemotherapy (single agent chlorambucil or doxorubicin-based combination chemotherapy), in addition to radiation to decrease relapse, has not been proven conclusively [J Clin Oncol 21 (11): 2115-22, 2003].

Traditionally, radiation therapy had been the primary treatment of patients with stage I or contiguous stage II aggressive non-Hodgkin's lymphoma (NHL). Radiation therapy alone can achieve long-term disease control within radiation fields in approximately 90% of treated patients. The dose of radiation ranges from 3500 to 5000 cGy and requires the use of megavoltage equipment. However, disease-free survival using radiation therapy alone is only 60% to 70% at 5 years [Ann Intern Med 104 (6): 747-56, 1986]. The success of combination chemotherapy in early-stage disease has led to combinations of chemotherapy and radiation therapy or to the use of chemotherapy alone [J Clin Oncol 11 (4): 720-5, 1993]. Two large randomized prospective trials document a better outcome with a combination of CHOP (cyclophosphamide+doxorubicin+vincristine+prednisone) and radiation therapy over CHOP alone [N Engl J Med 339 (1): 21-6, 1998]. The Southwest Oncology Group randomized 401 patients with localized aggressive NHL (stage I or II) to 3 cycles of CHOP plus involved-field radiation therapy or to 8 cycles of CHOP [N Engl J Med 339 (1): 21-6, 1998]. Overall survival at 5 years favored the combined modality arm (82% versus 72%, P=0.02). The Eastern Cooperative Oncology Group randomized 210 patients with bulky stage I and all stage II disease who had attained complete remission with 8 cycles of CHOP to radiation therapy or to no further treatment. With a median follow-up of 6 years, the disease-free survival favors the combined modality arm (73% versus 58%, P=0.03) with only marginal significance for overall survival (84% versus 70%, P=0.06). The British Columbia Cancer Agency treated 308 patients with early-stage diffuse large cell lymphoma using 3 cycles of doxorubicin-containing chemotherapy followed by involved-field radiation therapy; with a median follow-up of 7 years, the 10-year overall and progression-free survival rates were 80% and 63% respectively [J Clin Oncol 20 (1): 197-204, 2002].

Optimal treatment of advanced stages of low-grade lymphoma is controversial, and numerous clinical trials are in progress to settle treatment issues. Patients should be urged to participate. The reasons for controversy relate to the fact that the vast majority of patients with advanced stages of low-grade lymphoma are not cured with current therapeutic options. The rate of relapse is fairly constant over time, even in patients who have achieved complete responses to treatment. Indeed, relapse may occur many years after treatment. In this category, deferred treatment (watching carefully and waiting until the patient becomes symptomatic before initiating treatment) should be given consideration [Oncology 54 (6): 441-58, 1997; Lancet 362 (9383): 516-22, 2003]. Numerous prospective clinical trials of interferon alfa have shown no consistent benefit; the role for interferon in patients with indolent lymphoma remains controversial [J Clin Oncol 18 (10): 2007-9, 2000; J Immunother 24 (1): 58-65, 2001].

Standard therapy includes purine nucleoside analogs such as fludarabine or 2-chlorodeoxyadenosine [Blood 86 (5): 1710-6, 1995], oral alkylating agents (with or without steroids), or combination chemotherapy. Since none of these therapies are curative for advanced stage disease, innovative approaches are under clinical evaluation. These include intensive therapy with chemotherapy and total-body irradiation followed by autologous or allogeneic bone marrow or peripheral stem cell transplantation, the use of rituximab (anti-CD20 monoclonal antibody), and the use of radiolabeled monoclonal antibodies.

For patients with indolent, noncontiguous stage II and stage III lymphoma, central lymphatic irradiation has been proposed but is not usually recommended as a form of treatment [J Clin Oncol 11 (2): 233-8, 1993; Am J Clin Oncol 12 (3): 190-4, 1989].

Treatments of choice for patients with advanced stages of aggressive non-Hodgkin's lymphoma (NHL) are combination chemotherapy, either alone or supplemented by local-field irradiation [N Engl J Med 328 (14): 1023-30, 1993]. Doxorubicin-based combination chemotherapy produces long-term disease-free survival in 35% to 45% of patients [N Engl J Med 328 (14): 1002-6, 1993]. Higher cure rates have been reported in single-institution studies than in cooperative group trials.

A prospective randomized trial of 4 regimens (CHOP, ProMACE CytaBOM, m-BACOD, and MACOP-B) for patients with diffuse large cell lymphoma showed no difference in overall survival or time to treatment failure at 3 years [N Engl J Med 328 (14): 1002-6, 1993]. Other randomized trials have confirmed no advantage among the standard doxorubicin-based combinations versus CHOP [J Clin Oncol 12 (4): 769-78, 1994]. A randomized clinical trial failed to demonstrate a beneficial effect of adjuvant radiation therapy in advanced-stage aggressive NHL [J Clin Oncol 5 (9): 1329-39, 1987].

The combination of rituximab and CHOP has shown improvement in event-free survival (EFS) and overall survival (OS) compared to CHOP alone in 399 advanced stage patients over 60 years of age (EFS 57% versus 38%, P=0.002, and OS 70% versus 57%, P=0.0007, at 2 years) [N Engl J Med 346 (4): 235-42, 2002]. A trial of 635 patients aged 61 to 69 years with stage III/IV disease, elevated LDH, or performance status 2-4, randomized patients to CHOP or to ACVBP (intensified cyclophosphamide, doxorubicin, vindesine, bleomycin, prednisone with a consolidation phase). With a median follow-up of 68 months, patients who received ACVBP had superior event-free survival (39% versus 29% at 5 years, P=0.005) and overall survival (46% versus 38% at 5 years, P=0.036) [Blood 102 (13): 4284-9, 2003]. Two prospective randomized trials comparing CHOP to CNOP for patients aged 60 years and older with diffuse large cell lymphoma showed a significant advantage for CHOP in terms of disease-free and overall survival [Blood 101 (10): 3840-8, 2003]. Two other randomized trials of patients 70 years and older confirm the superiority of CHOP over other less toxic regimens in progression-free and overall survival [J Clin Oncol 16 (1): 27-34, 1998]. Although infusion regimens have been proposed, a randomized trial of infusional CHOP versus standard CHOP therapy showed no improvement in relapse-free or overall survival [J Clin Oncol 19 (3): 750-5, 2001]. Clinical trials continue to explore modifications of CHOP and rituximab with CHOP by increasing doses, reducing intervals between cycles, and combining new drugs with new mechanisms of action [Blood 102 (13): 4284-9, 2003; J Clin Oncol 21 (13): 2466-73, 2003; J Clin Oncol 21 (13): 2457-9, 2003].

Although considerable progress in the treatment of B-cell lymphoma has been observed in the past decade, there still remains plenty of room for improvements.

### SUMMARY OF THE INVENTION

This invention relates to a method of treating B-cell lymphoma comprising administering to a patient a chemotherapeutic regimen, followed by treatment with a radiolabeled anti-CD20 antibody, wherein at the time of said treatment with said radiolabeled antibody said patient is not refractory to said chemotherapeutic regimen and has not relapsed; typically, but not necessarily, at such time said patient will be one who has responsed to or is responding to said regimen.

The invention also relates to such a method wherein said patient has not previously been treated for said disease at the time of said chemotherapeutic regimen.

According to the invention, patients with B-cell lymphoma will be treated with up to six or more courses of conventional chemotherapy. These include, for example, CHOP (and modifications thereof), ICE, Mitoxantrone, Cytarabine, DVP, ATRA, Idarubicin, hoelzer chemotherapy regime, La La chemotherapy regime, ABVD, CEOP, 2-CdA, FLAG & IDA (with or without subsequent G-CSF treatment), VAD, M & P, C-Weekly, ABCM, MOPP, DHAP, etc. In addition, anti-CD20 antibodies (usually non-radiolabeled) could be administered as part of these regimens, although this is not mandatory. (See also column 3, lines 41-47 of U.S. Pat. No. 6,455,043.) The treatment of choice is the aforementioned combination of rituximab and CHOP.

After the last course of chemotherapy treatment, a radiolabeled anti-CD20 antibody is administered. The time point of administration relative to the end of the chemotherapy regimen may vary from one week to two years, preferably to nine months, most preferably to several weeks. In a preferred mode, the radiolabeled antibody is given approximately one week after the end of chemotherapy. Examples for radiolabeled antibodies are the commercially available drugs, Zevalin® and Bexxar®. However, the method is not restricted to the use of these antibodies. Any other antibody binding to the CD20 epitope and labelled with an isotope emitting alpha, beta or gamma rays may be utilized. The doses of the radiolabeled antibodies generally correspond to those used for the conventional monotherapy with these agents. A dose modification is not required. In special cases, the doses might be adjusted to the particular needs using conventional considerations.

For details of administration aspects of the therapies involved in this invention, see, e.g., U.S. Pat. No. 6,455,043, whose entire disclosure is incorporated by reference herein.

Aspects of the method of this invention not discussed in detail are fully conventional, such as B-cell lymphoma disease state definitions, conventional therapies therefor, determination of whether a patient is responding or refractory to a therapy, or has relapsed, etc. See, e.g., U.S. Pat. No. 6,455,043, among others. Known alternatives can also be employed, including antibody fragments for any antibody, any radioactive label other than those mentioned, etc. Trademarked products have the definitions given in the 2004 Physicians Desk Reference, whose disclosures are incorporated by reference herein.

The new regimen can be used for all types of B-cell lymphoma, including indolent and especially aggressive NHL, but it is not restricted to these examples.

The addition of the radiolabeled antibody in accordance with this invention will increase the response rate and the survival of the patients over the extent already achievable with the chemotherpy (+/−unlabelled anti-CD20 antibody) alone.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of the applications, patents and publications, cited herein are incorporated by reference herein.

### EXAMPLES

### Example 1

This example shows a protocol for a method of the present invention using ⁹⁰Y-ibritumomab tiuxetan (Zevalin®) for the treatment of 1^{st} line indolent NHL patients.

Study Design:

Phase III, randomized, multi-center trial

Patient Population:

Patients with histologically confirmed stage III or IV follicular non-Hodgkin's lymphoma (REAL classification) in CR (complete response) or PR (partial response) after first-line chemotherapy with or without Rituximab®, age 18 years or older

Exclusion Criteria
Any other anticancer treatment for NHL except the preceding first line chemotherapy
Prior radiation therapy
Prior myeloablative therapy
Patients who have not recovered from the toxic effects of the first line therapy
Any other malignancy or history of prior malignancy except non-melanoma skin tumors or stage 0 (in situ) cervical carcinoma
Presence of symptomatic CNS lymphoma
Patients with known HIV positivity
Patients with known seropositivity for HCV, HbsAG or other active infection uncontrolled by treatment
Patients with pleural effusion or ascites
Patients with abnormal liver function: total bilirubin>1.5×ULN or ALAT>2.5×ULN
Patients with abnormal renal function: serum creatinine>2.5×ULN
IgG<3 g/l
Presence of anti-murine antibody (HAMA) reactivity
Known hypersensitivity to murine antibodies or proteins
Immunotherapy during the preceding 6 months (including antibodies, interleukins, interferon maintenance—combination of first line chemotherapy with interferon is allowed)
Female patients who are pregnant or breast feeding, or adults of reproductive potential not employing an effective method of birth control during study treatment and for at least 12 months thereafter. Women of childbearing potential must have a negative serum pregnancy test at study entry.
Concurrent severe and/or uncontrolled medical disease (e.g. uncontrolled diabetes, congestive heart failure, myocardial infarction within 6 months prior to the study, unstable and uncontrolled hypertension, chronic renal disease, or active uncontrolled infection) which could compromise participation in the study
Patients who received any investigational drugs less than 4 weeks before entry in this study or who have not as yet recovered from the toxic effects of such therapy
Patients who underwent surgery within 4 weeks of entering the study or patients who have not as yet recovered from the side-effects of such treatment
Patients with a history of psychiatric illness or condition which could interfere with their ability to understand the requirements of the study (this includes alcoholism/drug addiction)
Patients unwilling or unable to comply with the protocol

Endpoints:
Progression-free survival, using two-sided stratified log-rank test
Change of response status (PR turning into CR), descriptive
Change in molecular response status, descriptive overall survival, using two-sided stratified log-rank test

Treatment Schedule:

Patients are randomized to receive either no treatment or an infusion of Rituximab at 250 mg/m² followed one week later by a single dose of ⁹⁰Y-ibritumomab tiuxetan 14.8 MBq/kg (0.4 mCi/kg) preceded by 250 mg/m² Rituximab®.

Patients randomized to the ⁹⁰Y-ibritumomab tiuxetan arm of this protocol will receive two infusions of 250 mg/m² Rituximab® one week apart. The first Rituximab® infusion will be given alone or in combination with 185 MBq (5 mCi) ¹¹¹In-ibritumomab tiuxetan for dosimetry or imaging. The second infusion of Rituximab®, administered one week later, will be followed immediately by 14.8 MBq/kg (0.4 mCi/Kg) of ⁹⁰Y-ibritumomab tiuxetan (max. 1184 MBq or 32 mCi) given as a slow intravenous push over 10 minutes.

Rituximab® should be administered intravenously through a dedicated line at an initial rate of 50 mg/hr. If hypersensitivity or infusion-related events do not occur, escalate the infusion rate in 50 mg/hr increments every 30 minutes, to a maximum of 400 mg/hr. If hypersensitivity or infusion-related events develop, the infusion should be temporarily slowed or interrupted. The patient should be treated according to the appropriate standard of care. The infusion can be continued at one-half the previous rate after symptoms have abated. Subsequent Rituximab® infusion can be administered at an initial rate of 100 mg/hr, and increased at 30 minute intervals by 100 mg/hr increments to a maximum of 400 mg/hr.

Whenever necessary, 185 MBq (5 mCi) of ¹¹¹In-ibritumomab tiuxetan will be used for radioimaging. The imaging dose of ¹¹¹In-ibritumomab tiuxetan will be administered by a 10-minute slow IV push injection immediately following the first infusion of Rituximab®. ¹¹¹In-ibritumomab tiuxetan may be directly infused by stopping the flow from the IV bag and injecting the radiolabeled antibody directly into the line. A 0.22-micron filter must be on line between the patient and the infusion port. Flush the line with at least 10 ml of normal saline after ¹¹¹In-ibritumomab tiuxetan has been infused.

Immediately following the second Rituximab® infusion, ⁹⁰Y-ibritumomab tiuxetan will be administered intravenously as a slow intravenous (i.v.) push over 10 minutes. ⁹⁰Y-ibritumomab tiuxetan may be directly infused by stopping the flow from the i.v. bag and injecting the radiolabeled antibody directly into the line. A 0.22 micron filter must be on line between the patient and the infusion port. Flush the line with at least 10 ml of normal saline after ⁹⁰Y-ibritumomab tiuxetan has been infused.

### Example 2

This example shows the schedule for the 1^{st} line treatment of patients with aggressive NHL.

Study Design:

Phase III, randomized, controlled multi-center trial

Patient Population:

Patients with histologically confirmed stage II, III or IV aggressive non-Hodgkin's lymphoma (REAL classification) in CR after first-line chemotherapy including Rituximab treatment, age 60 years or older

Exclusion Criteria:

See Example 1.

Endpoints:
Survival, using triangular (sequential) testing

Treatment Schedule:

See example 1

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method of treating B-cell lymphoma comprising administering to a patient a chemotherapeutic regimen, followed by treatment with a radiolabeled anti-CD20 antibody, wherein at the time of said treatment with said radiolabeled antibody said patient is not refractory to said chemotherapeutic regimen and has not relapsed.

2. A method of claim 1, wherein said chemotherapeutic regimen comprises administration of a non-radiolabeled anti-CD20 antibody.

3. The method of claim 1, wherein said chemotherapy comprises CHOP, ICE, Mitoxantrone, Cytarabine, DVP, ATRA, Idarubicin, hoelzer chemotherapy regime, La La chemotherapy regime, ABVD, CEOP, 2-CdA, FLAG & IDA (with or without subsequent G-CSF treatment), VAD, M & P, C-Weekly, ABCM, MOPP, or DHAP.

4. The method of claim 1, wherein said radiolabeled anti-CD20 antibody is administered from about one week to about two years after said chemotherapeutic regimen.

5. The method of claim 1, wherein said radiolabeled anti-CD20 antibody is administered from about one week to about nine months after said chemotherapeutic regimen.

6. The method of claim 1, wherein said radiolabeled anti-CD20 antibody is administered about one week after said chemotherapeutic regimen.

7. The method of claim 2, wherein said anti-CD20 antibody is a chimeric anti-CD20 antibody.

8. The method of claim 7, wherein said anti-CD20 antibody is Rituximab®.

9. The method of claim 7, wherein said radiolabeled anti-CD20 antibody is Zevalin®.

10. The method of claim 1, wherein said radiolabeled anti-CD20 antibody is Bexxar®.

11. The method of claim 1, wherein said B-cell lymphoma is low grade/follicular non-Hodgkin's lymphoma (NHL), small lymphocytic (SL) NHL, intermediate grade/follicular NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma or Waldenström's Macroglobulinemia.

12. A method of claim 1 wherein said patient has not previously been treated for said disease at the time of said chemotherapeutic regimen administration.

13. A method of claim 1 wherein at the time of said treatment with said radiolabeled antibody said patient has responded to or is responding to said regimen.
